(19) United States
(12) Patent Application Publication
(10) Pub. No.: US 2024/0058253 A1
(43) Pub. Date: Feb. 22, 2024

(54) SOFT-FOCUS COSMETICS COMPOSITIONS AND METHODS FOR REDUCING THE APPEARANCE OF SKIN IMPERFECTIONS

(71) Applicant: ELC MANAGEMENT LLC, Melville, NY (US)

(72) Inventors: Tao ZHENG, Dix Hills, NY (US); Alaine RANIERI, Holtsville, NY (US); Isaac David COHEN, Brooklyn, NY (US)

(21) Appl. No.: 17/891,103

(22) Filed: Aug. 18, 2022

(51) Int. Cl. A61K 8/81; A61K 8/9789; A61K 8/9794; A61K 8/06; A61K 8/29; A61K 8/25; A61Q 19/08; A61K 8/02

(52) U.S. Cl. CPC A61K 8/8152; A61K 8/9789; A61K 8/9794; A61K 8/064; A61K 8/29; A61K 8/25; A61Q 19/08; A61K 8/025; A61K 2800/412; A61K 2800/43; A61K 2800/621; A61K 2800/592; A61K 2800/26

(57) ABSTRACT

A soft-focus cosmetic composition contains at least one natural oil, at least one blurring agent, and at least one water-in-oil emulsifier. The soft-focus cosmetic composition may be a water-in-oil emulsion, where at least one natural oil is in the external phase. The soft-focus cosmetic composition has good radiance. A method for reducing the appearance of skin imperfections by applying to skin surfaces a soft-focus composition comprising at least one natural oil, at least one blurring agent, and at least one water-in-oil emulsifier.

### FIELD OF THE DISCLOSURE

The present disclosure generally relates to soft-focus cosmetic compositions and methods for reducing the appearance of skin imperfections.

### BACKGROUND

The cosmetics industry strives to shift from silicones due to regulatory compliance requirements, consumer demands, and the needs to reduce environmental impacts. Due to many skincare benefits that natural oils can bring into the products, natural oils have been used as one of the main candidates to replace silicones in cosmetic products. However, entirely or partially replacing silicones with natural oils brings major technical challenges in soft-focus cosmetic products, because silicones have very different reflective indexes from what natural oils have.

The soft-focus cosmetic compositions are highly desired because they can achieve a natural skin appearance with less skin imperfections. Specifically, by scattering the incoming light to blur skin imperfections, the soft-focus cosmetic compositions can hide skin imperfections but still let natural skin features show, while the simple covering-up techniques give undesirable artificial appearance due to the loss of most natural skin features. The standard measurement of the soft-focus effect is recognized to be the diffuse reflectance. The higher the diffuse reflectance of a composition is, the better its soft-focus effect is.

The soft-focus cosmetic compositions typically contain particles and liquid carriers, where the particles have different refractive indexes from what the liquid carriers have. It is recognized in the cosmetic industry that the bigger the differences of refractive indexes between the particles and liquid carriers in the compositions are, the better the soft-focus effects could be. For example, many particles commonly used in soft-focus cosmetic compositions have refractive indexes around 1.46, and silicones used for liquid carriers in such compositions have refractive indexes around 1.38. Such a difference in refractive indexes result in desired soft-focus effects in cosmetic compositions.

However, when silicones are replaced by natural oils, natural oils have reflective indexes (around 1.46) very similar to particles commonly used in soft-focus cosmetic compositions, and makes it very challenging to achieve meaningful soft-focus effects in cosmetic compositions based on natural oils instead of silicones.

Another challenge with soft-focus cosmetic compositions is to balance soft-focus effects and radiance effects. While soft-focus effects can hide skin imperfections, too much of such effect may result in the low level of the skin radiance. Being viewed as an indication of the healthy skin, radiance is also highly desired by the consumers. The standard measurement of radiance is recognized to be the specular reflectance. The higher the specular reflectance of a composition is, the better its radiance is. The soft-focus cometic compositions with the good level of radiance effects are highly desired by consumers but have always been challenging to make.

The present disclosure is directed to a cosmetic composition with unexpected soft-focus effect, comprising at least one natural oil, at least one blurring agent, and at least one water-in-oil emulsifier. Surprisingly, a good level of the skin radiance may also be achieved in such soft-focus cosmetic compositions. The disclosure is also directed to a method for reducing the appearance of skin imperfections by using such soft-focus cosmetic compositions.

### SUMMARY

The invention is directed to a soft-focus cosmetic composition comprising at least one natural oil, at least one blurring agent, and at least one water-in-oil emulsifier.

The invention is further directed to a method for reducing the appearance of skin imperfections by applying to skin surfaces a composition comprising at least one natural oil, at least one blurring agent, and at least one water-in-oil emulsifier.

### DETAILED DESCRIPTION

As used herein, the singular forms “a,” “an,” and “the” include the plural reference unless the context clearly dictates otherwise.

As used herein, a sentence reciting a string of alternates has the meaning that a string of sentences are provided such that each given alternate is provided in a sentence by itself. For example, the sentence “The composition comprises A, B, or C” has the meaning of the following three separate sentences: “Alternatively, the composition comprises A. Alternatively, the composition comprises B. Alternatively, the composition comprises C.”

As used herein, “and/or” has the meaning of “and” or “or”. For example, “A and/or B” has the meaning of “A, B, or both A and B.”

As used herein, “skin imperfection” means any type of mark, spot, discoloration, or flaw that appears on the skin. A non-limiting example of the skin imperfection is wrinkles and fine lines.

As used herein, “soluble” means that the solute dissolves partially or fully (and preferably fully) in the solvent, and preferably forms an equilibrium concentration of at least about 0.001 mg/mL at 25° C. The “oil-soluble active” means that the active dissolves partially or fully (and preferably fully) in the natural oil, and preferably forms an equilibrium concentration of at least about 0.001 mg/mL at 25° C. The “water-soluble active” means that the active dissolves partially or fully (and preferably fully) in water, and preferably forms an equilibrium concentration of at least about 0.001 mg/mL at 25° C.

It has been unexpectedly discovered that soft-focus effects can be achieved in cosmetic compositions based on natural oils. The present disclosure relates to a soft-focus cosmetic composition comprising at least one natural oil and at least one blurring agent. The present disclosure also relates to a method for reducing the appearance of skin imperfections by using a soft-focus cosmetic composition comprising at least one natural oil and at least one blurring agent.

The method for reducing the appearance of skin imperfections may comprise steps of: a) identifying a skin zone in need of reducing the appearance of skin imperfections; b) topically applying to said skin zone a soft-focus cosmetic composition. Preferably, the effect of reducing the appearance of skin imperfections is instant upon the application of the soft-focus cosmetic composition.

The soft-focus cosmetic composition may be in the form of emulsion, cream, lotion, paste, suspension, powder, gel, cushion compact, hot pour, or aerosol. Preferably, the soft-focus cosmetic composition is in the form of emulsion, lotion, or gel. More preferably, the soft-focus cosmetic composition is in the form of emulsion. An emulsion is a colloidal system consisting of two liquid phases of oil and water, one of which (internal phase) is dispersed into the other (external phase). An emulsion may be stable or metastable. The most preferred form of the soft-focus cosmetic composition is the water-in-oil (W/O) emulsion. A W/O emulsion means an emulsion consisting of an aqueous phase (internal phase) dispersed into a continuous oil phase (external phase). The at least one natural oil is in the external phase of the water-in-oil emulsion. According to one preferred embodiment, the external phase of the W/O emulsion is mainly composed of natural oils. The W/O emulsion with actives may deliver water and oil-soluble line and wrinkle active ingredients, provide long-term line and wrinkle reduction, and provide more texture options. The W/O emulsion may keep pigments and film former in the external phase for true color appearance and long-wear performance.

While soft-focus cosmetics compositions may be provided as foundations, it should be appreciated that these compositions may be employed in other cosmetics including, but not limited to, concealers, oil gels, lip color, cushion compacts, hot pours, and/or crème eye shadow. In embodiments of the present disclosure, the composition may be provided as a regimen with daytime foundation and nighttime un-pigmented skin lotion, foundation with SPF30, a silicone-free foundation, and/or universal anti-aging actives/oil cassette for makeup active delivery.

According to one more alternative embodiment, the soft-focus cosmetic composition is substantially free of silicones. The “substantially free of silicones” means that the total weight of silicones present in the soft-focus cosmetic composition is less than 0.01%, based on the total weight of the soft-focus cosmetic composition.

The soft-focus cosmetic composition of this disclosure has a diffuse reflectance of at least 2.5. The soft-focus cosmetic composition may provide double benefits with a single foundation formula: high radiance and high blurring effect at the same time. Conventional foundation formulas use a high level of particles for blurring effect. However, this approach often results in a matte and dull look on skin. Embodiments of the present disclosure may provide a unique property with both a soft-focus effect and a high radiance look. The soft-focus cosmetic composition of this disclosure has a specular reflectance of at least 3.4.

Natural Oils

The natural oil may provide for deep delivery of oil-soluble active ingredients, healthy skin from within, and a healthy skin barrier. The natural oil may include plant oils, fermented plant oils, or mixtures thereof. A plant oil means an oil extracted from a plant or a part of a plant. The extraction method may be chemical and/or physical. Preferred extraction methods may be selected from the group consisting of solvent extraction, mechanical extraction, distillation, and combinations thereof. A fermented plant oil means a mixture resulted from the fermentation of a plant oil as the organic substrate. Fermentation is a metabolic process that produces chemical changes in organic substrates through the action of microorganisms.

The plants may be land plants that include, but are not limited to, liverworts, hornworts, mosses, and vascular plants. The preferred plants may be vascular plants. The more preferred plants may be angiosperms. The preferred angiosperms are Simmondsiaceae, Asteraceae, Arecaceae, Lauraceae, Oleaceae, Euphorbiaceae, Fabaceae, Rosaceae, and Sapotaceae. The more preferred angiosperms are jojoba (Simmonsdia chinesis), sunflower (Helianthus annuus), coconut (Cocos nucifera), avocado (Persea gratissima), olive (Olea europaea), argan (Argania spinosa), Safflower (Carthamus tinctorius), Kukui Nut (Aleurites moluccana), Soybean (Glycine max), and Apricot (Prunus armeniaca). The most preferred angiosperms are jojoba (Simmonsdia chinesis), sunflower (Helianthus annuus), coconut (Cocos nucifera), avocado (Persea gratissima), olive (Olea europaea), and argan (Argania spinosa). The parts of the plant may be selected from the group consisting of stems, leaves, roots, sprouts, flowers, seeds, kernels, twigs, flowers, fruits, bark, and mixtures thereof. Preferred parts of the plant are seeds, fruits, and/or flowers. More preferred parts of the plant are seeds and/or fruits.

The microorganisms may be selected from the group consisting of fungi, bacteria, and mixtures thereof. Preferably, the microorganisms may be selected from the group consisting of yeasts, molds, lactic acid bacteria, and mixtures thereof. Nonlimiting examples of yeasts are Debaryomyces, Kluyveromyces, Saccharomyces, and Geotrichum. Preferred yeasts are Saccharomyces. Nonlimiting examples of molds are Mucor, penicillium, and rhizopus. Preferred molds are penicillium. Nonlimiting examples of lactic acid bacteria are Enterococcus, Streptococcus, Leuconostoc, Lactobacillus, and Pediococcus. Preferred lactic acid bacteria are Lactobacillus.

Preferably, the soft-focus cosmetic composition comprises at least one natural oil selected from the group consisting of the plant oil and the fermented plant oil made from Simmondsiaceae, Asteraceae, Arecaceae, Lauraceae, Oleaceae, Sapotaceae, and mixtures thereof. More preferably, the soft-focus cosmetic composition comprises at least one natural oil selected from the group consisting of the plant oil and the fermented plant oil made from jojoba (Simmonsdia chinesis), sunflower (Helianthus annuus), coconut (Cocos nucifera), avocado (Persea gratissima), olive (Olea europaea), argan (Argania spinosa), and mixtures thereof. Most preferably, the soft-focus cosmetic composition comprises at least one natural oil selected from the group consisting of jojoba (Simmonsdia chinesis) seed oil, coconut (Cocos nucifera) oil, avocado (Persea gratissima) oil, argan (Argania spinosa) kernel oil, olive (Olea europaea) fruit oil, sunflower (Helianthus annuus) oil, and mixtures thereof.

The total weight of at least one natural oil may be present in the soft-focus cosmetic composition in an amount ranging from 0.5% to about 20%, preferably from about 1% to about 15%, more preferably from about 3% to about 10%, based on the total weight of the soft-focus cosmetic composition.

The at least one natural oil may comprise two or more natural oils. In some embodiments, the at least one natural oil comprises jojoba (Simmonsdia chinesis) seed oil and Argan (Argania spinosa) kernel oil, with the weight ratio of jojoba (Simmonsdia chinesis) seed oil to Argan (Argania spinosa) kernel oil at from about 0.1:1 to about 1:1, preferably at from about 0.3:1 to about 0.7:1, most preferably at about 0.5:1. In some embodiments, the at least one natural oil comprises jojoba (Simmonsdia chinesis) seed oil and coconut (Cocos nucifera) oil, with the weight ratio of jojoba (Simmonsdia chinesis) seed oil to coconut (Cocos nucifera) oil at from about 0.5:1 to about 1:0.5, preferably at from about 0.7:1 to about 1:0.7, most preferably at about 1:1. In some embodiments, the at least one natural oil comprises avocado (Persea gratissima) oil and Argan (Argania spinosa) kernel oil, with the weight ratio of avocado (Persea gratissima) oil to Argan (Argania spinosa) kernel oil at from about 10:1 to about 0.5:1, preferably at from about 5:1 to about 1:1, most preferably at about 2:1. In some embodiments, the at least one natural oil comprises olive (Olea europaea) fruit oil and avocado (Persea gratissima) oil, with the weight ratio of olive (Olea europaea) fruit oil to avocado (Persea gratissima) oil at from about 0.5:1 to about 1:0.5, preferably at from about 0.7:1 to about 1:0.7, most preferably at about 1:1. In some embodiments, the at least one natural oil comprises Sunflower (Helianthus annuus) oil and Argan (Argania spinosa) kernel oil, with the weight ratio of Sunflower (Helianthus annuus) oil to Argan (Argania spinosa) kernel oil at from about 0.05:1 to about 1:1, preferably at from about 0.1:1 to about 0.5:1, most preferably at about 0.375:1. Most preferably, the at least one natural oil is in a form of an oil mixture comprising jojoba (Simmonsdia chinesis) seed oil, coconut (Cocos nucifera) oil, avocado (Persea gratissima) oil, Argan (Argania spinosa) kernel oil, Olive (Olea europaea) Fruit Oil, and Helianthus annus (sunflower) oil. The oil mixture may comprise natural oils with the weight ratio of jojoba (Simmonsdia chinesis) seed oil seed oil to coconut (Cocos nucifera) oil to avocado (Persea gratissima) oil to Argan (Argania spinosa) kernel oil to olive (Olea europaea) fruit oil to sunflower (Helianthus annuus) oil at (from about 0.1 to about 1):(from about 0.1 to about 1):(from about 10 to about 0.5):1:(from about 10 to about 0.5):(from about 0.05 to about 1). More preferably, The oil mixture may comprise natural oils with the weight ratio of jojoba (Simmonsdia chinesis) seed oil seed oil to coconut (Cocos nucifera) oil to avocado (Persea gratissima) oil to Argan (Argania spinosa) kernel oil to olive (Olea europaea) fruit oil to sunflower (Helianthus annuus) oil at about (from about 0.3 to about 0.7):(from about 0.3 to about 0.7):(from about 5 to about 1):about 1:(from about 5 to about 1):(from about 0.1 to about 0.5).

The natural oil may contain triglycerides, free-fatty acids, tocopherols, sterols, stanols, phospholipids, waxes, squalene, phenolic compounds, or mixtures thereof. The natural oils have many anti-aging therapeutic benefits on skin, including anti-inflammatory and antioxidant activity, promotion of wound healing, and/or repair of skin barrier. Inclusion of natural oils also may enhance oil-soluble active delivery in cosmetics compositions. Due to the high partition coefficient (log P), the natural oils can easily permeate through lipid bilayers of skin cell membrane. The oil-soluble active ingredients can be carried over through stratum corneum into skin epidermis layer for better anti-aging efficacy performance.

Blurring Agents

Blurring agents are materials capable of blurring skin imperfections by manipulating and transmission and scattering of light from and into skin. Such blurring effect is also called soft-focus effect. A cosmetic composition having such soft-focus effect is a soft-focus cosmetic composition. Skin imperfections like wrinkles and fine lines are perceptible through the contrast of light and dark that accentuates even very subtle creases. Soft-focus cosmetic compositions may optically reduce the contrast and hide the skin imperfections. Blurring agents do not often produce whiteness but rather a natural translucent finish. To achieve a blurring effect with cosmetic compositions, a higher refractive index (RI) of particles may be needed as well as a bigger difference of RI between blurring agents and vehicles.

The blurring agent may comprise a particulate material. The particulate material may be in the form of solid, filled or hollow particles. The shape of the particles may be oblate, prolate, bladed, or equant. Preferably, the particle shape of the blurring agent is sphere or platelet. The particulate material may be composed of one or more substances. When the particulate material comprises at least two substances, the substances may be mixed by the technique selected from the group of blending, encapsulation, coating, and combinations thereof. The particulate material may have an average particle size of less than 50 μm, preferably less than 30 μm, more preferably less than 20 μm.

The blurring agent may be present in the soft-focus cosmetic composition in an amount ranging from 0.01% to about 20%, preferably from about 0.05% to about 15%, more preferably from about 0.1% to about 10%, based on the total weight of the soft-focus cosmetic composition.

The blurring agent may be selected from the group consisting of effect pigments, silica based materials, polymeric materials, and mixtures thereof. The preferred blurring agent may comprise at least one effect pigment, at least one silica based material, at least one polymeric material, or mixtures thereof. The more preferred blurring agent may comprise an effect pigment, a silica based material, and a polymeric material.

The blurring agent may comprises at least one pigment. The pigment is a material with color effects that is completely or nearly insoluble in the application medium. The pigment may be organic materials, inorganic materials, or mixtures thereof. The pigment may also be natural, synthetic, or mixtures thereof. The pigment may be categorized, based on its color effects, into white pigments, colored pigment, black pigments, and effect pigments.

The blurring agent may comprise at least one effect pigment. The effect pigment is a pigment that gives additional color effects when applied in an application medium. The effect pigment may comprise titanium dioxide. A new trend in the effect pigment industry is the use of the transparent substance that is capable of generating color effects completely or predominately by the phenomenon of interference of light. The effect pigment may comprise a transparent substance. The preferred transparent substance is transparent synthetic mica. The preferred effect pigment may comprise at least two substances. Preferably, the effect pigment comprises a coating substance coated onto a substrate substance. The preferred substrate substance is transparent. The more preferred substrate substance is transparent synthetic mica. Most preferably, the effect pigment comprises titanium dioxide coated onto a transparent synthetic mica substrate. The effect pigment may have an average particle size in the range from about 1 μm to about 50 μm, preferably from about 3 μm to about 30 μm, more preferably from about 6 μm to about 25 μm. The non-limiting examples of the effect pigment can be purchased from BASF under the tradename Chione HD Infinite White. These ingredients provide a bright clean color due to the transparency of the substrate. Snowfall White 5130D has a particle size in the range from 10 μm to 36 μm, density of 3.2 kg/L, and approximate bulk density of 9 lb/ft³, 14 g/100 cm³. HD Pixel White S1305 has a particle size in the range from 30 μm to 130 μm, a density of 3.1 kg/L, and an approximate bulk density of 3 lb/ft³ and 4.5 g/100 cm³. HD Infinite White 5130V has a particle size range from 6 μm to 22 μm, a density of 3.2 kg/L, and an approximate bulk density of 9 lb/ft³ and 15 g/100 cm³.

The effect pigment may be present in the soft-focus cosmetic composition in an amount ranging from 0% to about 10%, preferably from about 0.01% to about 8%, more preferably from about 0.1% to about 5%, based on the total weight of the soft-focus cosmetic composition.

The blurring agent may comprise at least one silica based material. The silica based material may comprise at least one substance. The preferred silica based material comprises at least two substances. The at least two substances in the silica based material may be mixed by using the technique selected from the group of blending, encapsulation, coating, and combinations thereof. Preferably, the silica based material comprises a spheric silica microsphere. More preferably, the silica based material comprises porous spheric silica microspheres. Most preferably, the silica based material comprises titanium dioxide particles encapsulated in the porous spheric silica microspheres. The titanium dioxide particle may have an average particle size in the range from about 5 nm to about 20 nm, more preferably from about 8 nm to about 15 nm, most preferably at about 12 nm. The porous spheric silica microspheres may have an average particle size in the range from about 1 μm to about 10 μm, more preferably from about 3 μm to about 7 μm, most preferably at about 5 μm. The weight ratio of the titanium dioxide particle to the porous spheric silica microsphere in the silica based material may be in the range from about 1:10 to about 1:1, preferably from about 1:5 to about 1:1, more preferably at about 1:3. The silica based material may be optionally surface treated. Preferably, the surface treated silica based material may be treated by alkyl silane. The surface treated silica based material may have a hydrophobic coating. The silica based material may have a refractive index of at least 1.3, preferably at least 1.5, more preferably at least 1.7. Nonlimiting examples of the silica based material can be purchased from Sunjin Beauty Science under the tradenames SH219 and SH219-AS.

The silica based material may be present in the soft-focus cosmetic composition in an amount ranging from 0% to about 10%, preferably from about 0.01% to about 8%, more preferably from about 0.1% to about 5%, based on the total weight of the soft-focus cosmetic composition. The blurring agent may comprise at least one polymeric material. Preferably, the polymeric material comprises the homopolymer or copolymer of styrene and/or derivatives thereof. More preferably, the polymeric material comprises the copolymer of styrene and acrylates. Most preferably, the polymeric material comprises styrene/acrylates copolymer. The styrene/acrylates copolymer may have a refractive index in the range from about 1.3 to about 1.8, preferably in the range from about 1.5 to about 1.7, more preferably at about 1.6.

The polymeric material may be in the form of solid or hollow particles. The shape of the particles may be oblate, prolate, bladed, or equant. Preferably, the particle shape of the polymeric material is sphere or platelet. The preferred form for the polymeric material is hollow sphere particles.

The preferred polymeric material comprises styrene/acrylates copolymer in the form of the hollow sphere particles. The more preferred polymeric material is in the form of hollow sphere particles wherein the styrene/acrylates copolymer forms the outer shell of the hollow sphere particle. The hollow inside may have a refractive index of about 1.0. The different refractive indexes of the outer shell and the hollow inside of the polymeric material may lead to light fraction. One nonlimiting example of the polymeric blurring agent can be purchased from Dow under the tradename Sunspheres.

The polymeric material may be present in the soft-focus cosmetic composition in an amount ranging from 0% to about 10%, preferably from about 0.01% to about 8%, more preferably from about 0.1% to about 5%, based on the total weight of the soft-focus cosmetic composition.

Water-In-Oil Emulsifier

Emulsifiers are materials that help two liquid mix. Structurally, emulsifiers typically have lipophilic parts and hydrophilic parts. The Hydrophilic-Lipophilic Balance (“HLB”) value of an emulsifier has been used in the field to indicate whether the hydrophilic or lipophilic character dominates the property of such emulsifier. Oil-in-water emulsifiers have high HLB values (from about 10 to about 18), indicating that these emulsifiers are hydrophilic and suitable for emulsifying fats or oils in water. Water-in-oil emulsifiers have low HLB values (from about 2 to about 8, preferably about 3 to about 7, more preferably about 4 to about 6), indicating that these emulsifiers are lipophilic and suitable for water-in-oil emulsions.

The water-in-oil emulsifier may be selected from the group consisting of sorbitan isostearate, sorbitan stearate, sorbitan olivate, sorbitan sesquioleate, cetyl PEG/PPG-10/1 dimethicone, polyglyceryl-3 diisostearate, lauryl PEG-9 polydimethylsiloxyethyl dimethicone, PEG-30 dipolyhydroxystearate, polyglyceryl-6 polyricinoleate, polyglyceryl-2 isostearate, disteardimonium hectorite, polyglyceryl-4 diisostearate/polyhydroxystearate/sebacate, and mixtures thereof. Preferably, the water-in-oil emulsifier may be selected from the group consisting of sorbitan isostearate, cetyl PEG/PPG-10/1 dimethicone, polyglyceryl-3 diisostearate, lauryl PEG-9 polydimethylsiloxyethyl dimethicone, PEG-30 dipolyhydroxystearate, sorbitan sesquioleate, polyglyceryl-6 polyricinoleate, polyglyceryl-2 isostearate, disteardimonium hectorite, and mixtures thereof.

The water-in-oil emulsifier may be present in the soft-focus cosmetic composition in an amount ranging from 0.01% to about 20%, preferably from about 0.05% to about 15%, more preferably from about 0.1% to about 10%, based on the total weight of the soft-focus cosmetic composition.

Additional Components

The soft-focus cosmetics composition may further contain other additional components, which may be selected by the artisan according to the desired characteristics of the final product and which are suitable for rendering the compositions more cosmetically or aesthetically acceptable or to provide them with additional usage benefits. The components useful herein are conveniently categorized by a certain benefit or their postulated mode of action, however, a given category is not limiting of its use. Further, it is understood the one component may provide multiple benefits.

Oil-Soluble Actives and Water-Soluble Actives

The oil-soluble active may comprise any active mentioned in the Additional Components section that dissolves partially or fully (and preferably fully) in the natural oil, and preferably forms an equilibrium concentration of at least about 0.001 mg/mL at 25° C., or mixtures thereof.

Preferably, the oil-soluble active may comprise sunflower (Helianthus annuus) seedcake, Hordeum vulgare (barley) extract, cucumic sativus (cucumber) fruit extract, Commiphora mukul resin extract, C12-15 alkyl benzoate, tribehenin, ceramide NG, PEG-10 phytosterol, palmitoyl hexapeptide-12, phospholipids, caprylic/capric triglyceride, tocopherol, stearic acid, oleic acid, linoleic acid, palmitic acid, phytosteryl canola glycerides, triolein, dipalmitoyl hydroxyproline, tocopheryl acetate, or mixtures thereof.

One nonlimiting example of the oil-soluble active is a complex of sunflower (Helianthus annuus) seedcake, Hordeum vulgare (barley) extract, and cucumic sativus (cucumber) fruit extract. This complex may function as a barrier. This complex may be purchase from Barnet under the tradename Phytofix-EC, which is a unique and optimized blend solubilized in Squalane containing soluble sphingolipids, triglycerides and sterols to mimic the structure of the skin's membrane. One nonlimiting example of the oil-soluble active is Commiphora mukul resin extract, which may function to address lines and wrinkles. it is a resin extract obtained from a tree found in the arid parts of the North-West India, Commiphora mukul. It locally increases adipocyte size to plump the skin from within. Thanks to its “volumizing” effect, it restores volume of the skin and soften wrinkles. One more nonlimiting example of the oil-soluble active is Palmitoyl Hexapeptide-12, which may function to address lines and wrinkles. It is an anti-aging, wrinkle smoothing and cutaneous barrier repair ingredient. It stimulates cell communication and then repairs the age-related skin damage.

The oil-soluble active may be present in an amount ranging from 0.1% to about 30%, preferably from about 1% to about 20%, more preferably from about 2% to about 15%, based on the total weight of the soft-focus cosmetic composition.

The water-soluble active may comprise any active mentioned in the Additional Components section that dissolves partially or fully (and preferably fully) in water, and preferably forms an equilibrium concentration of at least about 0.001 mg/mL at 25° C., or mixtures thereof.

The water-soluble active may comprise niacinamide, trehalose, acetyl hexapeptide-8, hyaluronic acid, sucrose, or mixtures thereof. Niacinamide and/or hyaluronic acid may function for moisturizing. Trehalose may function for moisturizing. Acetyl hexapeptide-8 may function to address lines and wrinkles. Sucrose may function for moisturizing.

The water-soluble active may be present in the soft-focus cosmetic composition in an amount ranging from 0.1% to about 30%, preferably from about 1% to about 20%, more preferably from about 2% to about 15%, based on the total weight of the soft-focus cosmetic composition.

Anti-Oxidants and Radical Scavengers

Anti-oxidants and radical scavengers are especially useful for providing protection against UV radiation which can cause increased scaling or texture changes in the stratum corneum and against other environmental agents which can cause skin damage. Such anti-oxidants/radical scavengers include, for example, tocopherol sorbate and other esters of tocopherol, and tocopherol sorbate.

Anti-Inflammatory Agents

Anti-inflammatory agents enhance the skin appearance benefits, by for example, contribution of uniformity and acceptable skin tone and/or color. Optionally, the anti-inflammatory agent includes a steroidal anti-inflammatory agent and a non-steroidal anti-inflammatory agent. The steroidal anti-inflammatory agent may be hydrocortisone. So-called “natural” anti-inflammatory agents are also useful. For example, alpha bisabolol, aloe vera, Manjistha (extracted from plants in the genus Rubia, particularly Rubia Cordifolia), and Guggal (extracted from plants in the genus Commiphora, particularly Commiphora Mukul), kola extract, chamomile, and sea whip extract, may also be used.

Antimicrobial Agents

As used herein, “antimicrobial agents” means a compound capable of destroying microbes, preventing the development of microbes or preventing the pathogenic action of microbes. Antimicrobal agents are useful, for example, in controlling acne. Preferred antimicrobial agents are benzoyl peroxide, erythromycin, tetracycline, clindamycin, azelaic acid, sulfur resorcinol, phenoxyethanol, and Irgasan™ DP 300 (Ciba Geigy Corp., U.S.A.). A safe and effective amount of an antimicrobial agent may be added to emulsions herein, preferably from 0.001% to 10%, more preferably from 0.01% to 5%, still more preferably from 0.05% to 2%.

Chelators

As used herein, “chelator” refers to a compound that reacts for removing a metal ion from a system by forming a complex so that the metal ion cannot readily participate in or catalyze chemical reactions. The inclusion of a chelator is especially useful for providing protection against UV radiation which can contribute to excessive scaling or skin texture changes and against other environmental agents which can cause skin damage. Exemplary chelators that are useful herein are disclosed in U.S. Pat. No. 5,487,884, Bissett et al, issued Jan. 30, 1996; PCT application 91/16035 and 91/16034, Bush et al, published Oct. 31, 1995. Preferred chelators are furildioxime and derivatives thereof.

Silicone Elastomers

The compositions may include a non-emulsifying crosslinked organopolysiloxane elastomer. The term “non-emulsifying,” as used herein, defines crosslinked organopolysiloxane elastomers from which polyoxyalkylene units are absent. Such elastomers are used to reduce the tackiness/stickiness feel associated with skin conditioning agents.

The elastomers may be dimethicone/vinyl dimethicone crosspolymers, vinyl dimethicone/lauryl dimethicone crosspolymers, C30-C45, alkyl ceteayl dimethicone/polycyclohexane oxide crosspolymers, and mixtures thereof.

Dimethicone/vinyl dimethicone crosspolymers are supplied by a variety of suppliers including Dow Corning (DC 9040 and DC 9041), General Electric (SFE 839), Shin Etsu (KSG-15, 16, 18 [dimethicone/phenyl vinyl dimethicone crosspolymer]), and Grant Industries (GRANSIL™ line of elastomers). Cross-linked organopolysiloxane elastomers and processes for making them are further described in U.S. Pat. No. 4,970,252 to Sakuta, et al., issued Nov. 13, 1990; U.S. Pat. No. 5,760,116 to Kilgour, et al., issued Jun. 2, 1998; U.S. Pat. No. 5,654,362 to Schulz, Jr., et al. issued Aug. 5, 1997.

The vinyl dimethicone/lauryl dimethicone crosspolymers include vinyl dimethicone/lauryl dimethicone crosspolymer & mineral oil (tradename KSG-41); vinyl dimethicone/lauryl dimethicone crosspolymer & isododecane (tradename KSG-42); vinyl dimethicone/lauryl dimethicone crosspolymer & triethylhexanoin (tradename KSG-43); vinyl dimethicone/lauryl dimethicone crosspolymer & squalane (tradename KSG-44). Each of these “KSG” denominated silicone elastomers is available from Shinestu Chemical.

Commercially available cyclomethicone and C30-C45 alkyl ceteayl dimethicone/polycyclohexane oxide crosspolymer is available from GE Silicone under the tradename Velvasil 125.

Whitening Agents

The compositions herein may further comprise from 0.001% to 10%, or from 0.1% to 5% of a whitening agent. Nonlimiting examples of suitable whitening agents are those which are compatible with aqueous compositions. The whitening agents may include active ingredients that not only alter the appearance of the skin, but also improve hyperpigmentation as compared to pre-treatment.

Useful whitening agents may include ascorbic acid compounds, azelaic acid, butyl hydroxy anisole, gallic acid and its derivatives, glycyrrhizinic acid, hydroquinoine, kojic acid, arbutin, mulberry extract, and mixtures thereof. Use of combinations of whitening agents is believed to be advantageous in that they may provide whitening benefit through different mechanisms.

The ascorbic acid compound may be an ascorbic acid salt or derivative thereof. Exemplary water-soluble salt derivatives include, but are not limited to, L-ascorbic acid 2-glucoside, L-ascorbyl phosphate ester salts such as sodium L-ascorbyl phosphate, potassium L-ascorbyl phosphate, magnesium L-ascorbyl phosphate, calcium L-ascorbyl phosphate, aluminum L-ascorbyl phosphate. L-ascorbyl sulfate ester salts can also be used. Examples are sodium L-ascorbyl sulfate, potassium L-ascorbyl sulfate, magnesium L-ascorbyl sulfate, calcium L-ascorbyl sulfate and aluminum L-ascorbyl sulfate.

PH Adjusters

The compositions may further comprise a pH adjuster to control the pH of the composition. Particularly, the pH of the composition of the present disclosure is within the range of from about 5 to about 8, or from about 5.2 to about 7.8, or from about 5.4 to about 7.6, for example about 5.4, about 5.6, about 5.8, about 6.0, about 6.2, about 6.4, about 6.6, about 6.8, about 7.0, about 7.2, about 7.4, about 7.6 and any ranges therebetween.

The compositions may further comprise from about 0.01% to about 5%, or from about 0.1% to about 3%, or from about 0.3% to about 2%, or from about 0.4% to about 1.8%, and or from about 0.5% to about 1.6%, for example about 0.4%, about 0.5%, about 0.6%, about 0.7%, about 0.8%, about 0.9%, about 1.0%, about 1.1%, about 1.2%, about 1.3%, about 1.4%, about 1.5%, about 1.6% and any ranges therebetween, by weight of the composition, of a pH adjuster, wherein said pH adjuster is selected from the group consisting of potassium hydroxide, sodium hydroxide, ammonium hydroxide, aminomethyl propanol, triethanolamine, tetrahydroxypropyl ethylene diamine and any combinations thereof.

When a polymeric emulsifier and a pH adjuster is used, it may be desired to optimize the ratio of the polymeric emulsifier to the pH adjuster. For example, the weight ratio of the polymeric emulsifier to the pH adjuster may be between about 1:5 and about 1:0.5, or between about 1:3 to about 1:1, for example about 1:3, about 1:2.5, about 1:2, about 1:1.5, about 1:1.1 and any ranges therebetween.

Thickeners

The compositions may further comprise a thickener (also called a thickening agent) or an additional thickener if the emulsifier in the composition also functions as a thickener. The compositions may comprise from about 0.1% to about 5%, or, alternatively, from about 0.2% to about 2%, of a thickener or an additional thickener when present. Suitable classes of thickeners include but are not limited to carboxylic acid polymers, polyacrylamide polymers, sulfonated

polymers, copolymers thereof, hydrophobically modified derivatives thereof, and mixtures thereof.

The thickener may be an acrylate cross linked silicone copolymer network (also sometimes referred to as “polyacrylate siloxane copolymer network”). Suitable thickening agents may also generally include carboxylic acid polymers, polyacrylamide polymers or copolymers, sulfonated polymers, gum, clays, cellulose or modified cellulosic compositions, and the like.

Other Components

In addition to the above described components, the compositions herein may further include preservatives and preservative enhancers such as water-soluble or solubilizable preservatives including Germall 115, methyl, ethyl, propyl and butyl esters of hydroxybenzoic acid, benzyl alcohol, imidazolidinyl urea, EDTA and its salts, Bronopol (2-bromo-2-nitropropane-1,3-diol) and phenoxypropanol; antifoaming agents; binders; biological additives; bulking agents; coloring agents; essential oils and solubilizers thereof; other natural extracts; compounds which stimulate collagen production; yeast fermented filtrates, and others.

### EXPERIMENTS

### Example 1

A oil mixture was made by mixing at room temperature 5 parts of jojoba (Simmonsdia chinesis) seed oil seed oil, 5 parts of coconut (Cocos nucifera) oil, 20 parts of avocado (Persea gratissima) oil, 10 parts of Argan (Argania spinosa) kernel oil, 20 parts of olive (Olea europaea) fruit oil, and 3.75 parts of sunflower (Helianthus annuus) oil, until all dissolved. All parts were based on weight.

### Example 2

A cosmetic base (Sample 1) was made by mixing 7 parts of the oil mixture of Example 1 and 50 parts of water. Cosmetic compositions were made by adding 5 parts of particulate materials (as shown in Table 1) to 95 parts of the cosmetic base, and mixing for approximately 15 mins to complete uniformly and completely dispersed. All parts were based on weight.

A spectral reflectance test using a spectrophotometer (KONICA MINOLTA CM700D Spectrophotometer) was performed on the resulting cosmetic compositions. The sample was uniformly applied to PMMA Helioplate HD6 with finger and moderate force in circle and cross spreading pattern within 1 minute. The finger was cleaned with alcohol and pre-saturated with sample. The applied dosage was 2 mg/cm² recommended by FDA. The prepared sample was let dry in room temperature for 20 minutes before the test.

After calibration of KONICA MINOLTA CM700D Spectrophotometer, the instrument was set to reflectance spectrum mode with both Specular Component Included (SCI) and Specular Component Excluded (SCE). A 8 mm target mask was attached to the instrument for larger measurement area. The visible light spectrum between 400 nm and 700 nm was selected for full color spectral analysis.

The prepared sample was placed on the black side of the Leneta chart card for reflectance test to eliminate the light reflection from the Leneta card background. Both diffused reflectance and total reflectance were measured. The diffused reflectance data was used as blurring indicator, because it captures all light scattered by the cosmetic film from 0 to 180 degree angle. To compare the blurring effect, the diffused reflectance at peak of spectrum was used. For example, for liquid foundation cosmetics, the reflectance at 620 nm wavelength was used to represent the foundation shade.

TABLE 1
Soft-focus effects of cosmetic compositions with different particulate materials.

| Sample No. | Particulate Material | Diffuse Reflectance |
|---|---|---|
| Sample 1 | None | 2.13 |
| Sample 2 | Prizmalite (INCI: Soda Lime Glass Beads) | 3.08 |
| Sample 3 | Sunspheres (INCI: styrene/acrylates copolymer, | 5.42 |
| | purchased from Dow) | |
| Sample 4 | SH219 (INCI: Silica/Titanium Dioxide, purchased | 4.56 |
| | from Sunjin Beauty Science) | |
| Sample 5 | SH219-AS (INCI: Silica/Titanium | 4.68 |
| | Dioxide/Triethoxycaprylylsilane, purchased | |
| | from Sunjin Beauty Science) | |
| Sample 6 | Chione HD Infinite White S130v (INCI: Synthetic | 11.39 |
| | Fluorphlogopite/Titanium dioxide, Tin Oxide, | |
| | purchased from BASF) | |
| Sample 7 | Monoveil (INCI: Silica) | 3.58 |
| Sample 8 | Barrium Sulfate (INCI: Barium Sulfate) | 2.69 |
| Sample 9 | Kaolin (INCI: Kaolin) | 2.88 |
| Sample 10 | Gransil SIW-SBF (INCI: Isododecane/Dimethicone/ | 1.87 |
| | Polysilicone-11/Coco-Caprylate\Caprate/Water\Aqual\Eau/ | |
| | Butylene Glycol, purchased from Grant Industries) | |
| Sample 11 | Gransil EP-LS (INCI: Polysilicone-11/Laureth-12, purchased | 3.26 |
| | from Grant Industries) | |

As shown in Table 1, Samples 3 (Sunspheres), 4 (SH219), 5 (SH219-AS), and 6 (Chione HD Infinite White S130v) provided unexpected high diffuse reflectance (much higher than 2.5), so unexpected soft-focus effects were achieved with these compositions based on natural oils.

### Example 3

A cosmetic base (Sample 1) was made by mixing 7 parts of the oil mixture of Example 1 and 50 parts of water. Cosmetic compositions were made, according to weight percentages of Table 2, by adding particulate materials to the cosmetic base, and mixing for approximately 15 mins to complete uniformly and completely dispersed. All parts were based on weight. The particular material A is Chione HD Infinite White S130v (INCI: synthetic fluorphlogopite/titanium dioxide, purchased from BASF); B is SH219 (INCI: Silica/Titanium Dioxide, purchased from Sunjin Beauty Science); and C is Sunspheres (INCI: styrene/acrylates copolymer, purchased from Dow).

A spectral reflectance test using a spectrophotometer (KONICA MINOLTA CM700D Spectrophotometer) was performed on the resulting cosmetic compositions. The sample was uniformly applied to PMMA Helioplate HD6 with finger and moderate force in circle and cross spreading pattern within 1 minute. The finger was cleaned with alcohol and pre-saturated with sample. The applied dosage was 2 mg/cm2 recommended by FDA. The prepared sample was let dry in room temperature for 20 minutes before the test.

After calibration of KONICA MINOLTA CM700D Spectrophotometer, the instrument was set to reflectance spectrum mode with both Specular Component Included (SCI) and Specular Component Excluded (SCE). A 8 mm target mask was attached to the instrument for larger measurement area. The visible light spectrum between 400 nm and 700 nm was selected for full color spectral analysis.

The prepared sample was placed on the black side of the Leneta chart card for reflectance test to eliminate the light reflection from the Leneta card background. Both diffused reflectance and total reflectance were measured. The specular reflectance was calculated based on the equation (Reflectanceₜₒₜₐₗ=Reflectance_{Diffused}+Reflectance_{Specular}). The diffused reflectance data was used as blurring indicator, because it captures all light scattered by the cosmetic film from 0 to 180 degree angle. To compare the blurring effect, the diffused reflectance at peak of spectrum was used. For example, for liquid foundation cosmetics, the reflectance at 620 nm wavelength was used to represent the foundation shade.

TABLE 2
Soft-focus and radiance effects of cosmetic compositions
with different blurring agents at various percentages.

| | A | B | C | Diffuse | Specular |
|---|---|---|---|---|---|
| Sample No. | (%) | (%) | (%) | reflectance | Reflectance |
| Sample 1 | 0 | 0 | 0 | 2.35 | 8.14 |
| Sample 12 | 0.5 | 0 | 0 | 3.77 | 8.08 |
| Sample 13 | 5 | 0 | 0 | 11.2 | 5.78 |
| Sample 14 | 10 | 0 | 0 | 21.4 | 4.89 |
| Sample 15 | 0 | 0.5 | 0 | 2.75 | 7.74 |
| Sample 16 | 0 | 5 | 0 | 5.56 | 4.91 |
| Sample 17 | 0 | 10 | 0 | 7.65 | 3.48 |
| Sample 18 | 0 | 0 | 0.5 | 2.62 | 7.94 |
| Sample 19 | 0 | 0 | 5 | 5.77 | 6.65 |
| Sample 20 | 0 | 0 | 10 | 10.23 | 5.7 |
| Sample 21 | 0.167 | 0.167 | 0.167 | 2.97 | 8.09 |
| Sample 22 | 1.67 | 1.67 | 1.67 | 7.55 | 5.69 |
| Sample 23 | 3.33 | 3.33 | 3.33 | 12.89 | 4.18 |

As shown in Table 2, all samples provided unexpected high diffuse reflectance (higher than 2.5) and high specular reflectance (higher than 3.4), so unexpected instant soft-focus effects and radiance effects were achieved with these compositions based on natural oils.

### Example 3

A cosmetic base (Sample 1) was made by mixing 7 parts of the oil mixture of Example 1 and 50 parts of water. A soft-focus cosmetic (Sample 24) composition was made as following:

The Sequence 1 materials were mixed and heated up to 80° C. in a homogenizer at 5,000 RPM. The prepared Sequence 2 oil were added into the Sequence 1 mixture and mixed well. The Sequence 3 water was heated to 75° C. and then the Sequence 4 materials were added into water and mixed well. The combined Sequence 3 and 4 was transferred into the combined Sequence 1 and 2 under homogenizer increased to 8,000 RPM, and then cooled to 30° C. to form the cosmetic base. The soft-focus cosmetic composition (Sample 24) was made by adding Sequence 5 materials into the cosmetic base, and mixing for approximately 15 mins to complete uniformly and completely dispersed.

TABLE 3
A soft-focus cosmetic composition.

| Sequence | category | Ingredients | wt. % |
|---|---|---|---|
| 1 | Blurring agents | TERRA WHITE-NJE5¹ (INCI: MICA/Titanium | 5 |
| | | Dioxide (CI 77891)/Jojoba Esters) | |
| | | RBTD-671-11S2² (INCI: Titanium Dioxide (CI 77891)/ | 6.667 |
| | | Triethoxycaprylylsilane/Alumina/Silica) | |
| | | Barium Sulfate | 3.5 |
| 2 | Water-in-oil emulsifier | PEG-30 Dipolyhydroxystearate | 1.5 |
| 3 | Natural oil | The oil mixture of Example 1 | 6.375 |
| | Oil-soluble actives | Caprylic/Capric Triglyceride | 2 |
| 4 | Water | Water | Q.S. |
| | | Niacinamide | 1 |
| | | Glycerin | 1 |
| | | Trehalose | 0.5 |
| | | Sucrose | 0.2 |
| | | Potassium Sorbate | 0.1 |
| 5 | Water-soluble actives | Citric Acid | 0.08 |
| | | Disodium EDTA | 0.05 |
| | | Hyaluronic Acid, Sodium Salt | 0.01 |
| | | Acetyl Hexapeptide-8 | 0.0005 |
| | | Palmitoyl Hexapeptide-12 | 0.0004 |
| | | Palmitoyl Tripeptide-1 | 0.0002 |
| 6 | Blurring agents | Gransil SIW-SBF³ (INCI: Isododecane/Dimethicone/ | |
| | | Polysilicone-11/Cococaprylate\Caprate/Water/ | 1 |
| | | Butylene Glycol), | |
| 7 | Blurring agents | Chione HD Infinite White S130v⁴ | 0.2 |
| | | Gransil EP-LS⁵ (INCI: Polysilicone-11/Laureth-12) | 2.5 |

¹Purchased from Sun Chemical under the trade name TERRA WHITE-NJE5
²Purchased from Kobo Products, Inc. under the trade name RBTD-671-11S2
³Purchased from Grant Industries under the trade name Gransil SIW-SBF
⁴Purchased from BASF under the trade name Chione HD Infinite White S130v
⁵Purchased from Grant Industries under under the trade name Gransil EP-LS

Comparable examples were commercial products purchased on the market with known soft-focus efficacy: Lancome Rénergie Lift Makeup Foundation SPF 27 (Sample 25), IT Cosmetics CC+ Cream with SPF 50+(Sample 26), and Tarte Amazonian Clay 16-Hour Full Coverage Foundation (Sample 27).

A spectral reflectance test using a spectrophotometer (KONICA MINOLTA CM700D Spectrophotometer) was performed on the resulting cosmetic compositions. The sample was uniformly applied to PMMA Helioplate HD6 with finger and moderate force in circle and cross spreading pattern within 1 minute. The finger was cleaned with alcohol and pre-saturated with sample. The applied dosage was 2 mg/cm2 recommended by FDA. The prepared sample was let dry in room temperature for 20 minutes before the test.

After calibration of KONICA MINOLTA CM700D Spectrophotometer, the instrument was set to reflectance spectrum mode with both Specular Component Included (SCI) and Specular Component Excluded (SCE). A 8 mm target mask was attached to the instrument for larger measurement area. The visible light spectrum between 400 nm and 700 nm was selected for full color spectral analysis.

The prepared sample was placed on the black side of the Leneta chart card for reflectance test to eliminate the light reflection from the Leneta card background. Both diffused reflectance and total reflectance were measured. The specular reflectance was calculated based on the equation (Reflectanceₜₒₜₐₗ=Reflectance_{Diffused}+Reflectance_{Specular}). The diffused reflectance data was used as blurring indicator, because it captures all light scattered by the cosmetic film from 0 to 180 degree angle. To compare the blurring effect, the diffused reflectance at peak of spectrum was used. For example, for liquid foundation cosmetics, the reflectance at 620 nm wavelength was used to represent the foundation shade.

TABLE 4
Comparing with commercial benchmarks.

| | Diffuse | Specular |
|---|---|---|
| Sample | reflectance | Reflectance |
| Sample 24 | 33.40 | 3.79 |
| Sample 25 | 29.77 | 2.79 |
| Sample 26 | 41.19 | 2.37 |
| Sample 27 | 25.09 | 1.14 |

As shown in Table 4, the soft-focus cosmetic composition (Sample 24) showed higher specular reflectance (at 3.79) than benchmarks while still maintained very high soft-focus effect (with diffuse reflectance at 33.40, much higher than required 2.5).

Although the present disclosure and its advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A soft-focus cosmetic composition comprising at least one natural oil, at least one blurring agent, and at least one water-in-oil emulsifier, wherein the soft-focus cosmetic composition has a diffuse reflectance of at least 2.5.

2. The soft-focus cosmetic composition of claim 1, wherein the at least one natural oil is selected from the group consisting of plant oil, fermented plant oil, and mixtures thereof.

3. The soft-focus cosmetic composition of claim 2, wherein the at least one natural oil is selected from the group consisting of the plant oil and the fermented plant oil made from Simmondsiaceae, Asteraceae, Arecaceae, Lauraceae, Oleaceae, Sapotaceae, and mixtures thereof.

4. The soft-focus cosmetic composition of claim 1, wherein the at least one natural oil is present in the soft-focus cosmetic composition in an amount ranging from about 0.5% to about 20%, based on the total weight of the soft-focus cosmetic composition.

5. The soft-focus cosmetic composition of claim 1, wherein the at least one blurring agent comprises a particulate material having an average particle size of less than 50 μm.

6. The soft-focus cosmetic composition of claim 1, wherein the at least one blurring agent comprises an effect pigment.

7. The soft-focus cosmetic composition of claim 7, wherein the effect pigment comprises transparent synthetic mica.

8. The soft-focus cosmetic composition of claim 8, wherein the effect pigment comprises titanium dioxide coated onto a transparent synthetic mica substrate.

9. The soft-focus cosmetic composition of claim 7, wherein the effect pigment is present in an amount ranging from 0% to about 10%, based on the total weight of the soft-focus cosmetic composition.

10. The soft-focus cosmetic composition of claim 1, wherein the at least one blurring agent comprises a silica based material.

11. The soft-focus cosmetic composition of claim 11, wherein the silica based material comprises porous spheric silica microspheres.

12. The soft-focus cosmetic composition of claim 12, wherein the silica based material comprises titanium dioxide particles encapsulated in porous silica microspheres.

13. The soft-focus cosmetic composition of claim 13, wherein the weight ratio of the titanium dioxide particles to the porous spheric silica microspheres in the silica based material is in the range from about 1:10 to about 1:1.

14. The soft-focus cosmetic composition of claim 11, wherein silica based material is present in an amount ranging from 0% to about 10%, based on the total weight of the soft-focus cosmetic composition.

15. The soft-focus cosmetic composition of claim 1, wherein the at least one blurring agent comprises a polymeric material.

16. The soft-focus cosmetic composition of claim 16, wherein the polymeric material comprises styrene/acrylates copolymer.

17. The soft-focus cosmetic composition of claim 17, wherein the polymeric material comprises styrene/acrylates copolymer is in the form of the hollow sphere particles.

18. The soft-focus cosmetic composition of claim 16, wherein the polymeric material is present in an amount ranging from 0% to about 10%, based on the total weight of the soft-focus cosmetic composition.

19. The soft-focus cosmetic composition of claim 16, wherein the at least one water-in-oil emulsifier has a HLB value from about 2 to about 8.

20. The soft-focus cosmetic composition of claim 1, wherein the soft-focus cosmetic composition is a water-in-oil emulsion, wherein the at least one natural oil is in the external phase of the water-in-oil emulsion.

21. The soft-focus cosmetic composition of claim 1, wherein the soft-focus cosmetic composition has a specular reflectance of at least 3.4.

22. A soft-focus cosmetic composition comprising:
at least one natural oil present in an amount ranging from about 0.5% to about 20%;
at least one blurring agent present in an amount ranging from 0.01% to about 20%, wherein the at least one blurring agent is selected from the group consisting of effect pigment, silica based material, polymeric material, and mixtures thereof;
at least one water-in-oil emulsifier present in an amount ranging from about 0.01% to about 20%;
wherein all percentages are based on the total weight of the soft-focus cosmetic composition;
wherein the soft-focus cosmetic composition has a diffuse reflectance of at least 2.5;
wherein the soft-focus cosmetic composition has a specular reflectance of at least 3.4;
wherein the soft-focus cosmetic composition is a water-in-oil composition.

23. A method for reducing the appearance of skin imperfections, comprising:
a) identifying an skin zone in need of reducing the appearance of skin imperfections;
b) topically applying to said skin zone a soft-focus cosmetic composition, wherein the soft-focus cosmetic composition comprising at least one natural oil, at least one blurring agent, and at least one water-in-oil emulsifier, wherein the soft-focus cosmetic composition has a diffuse reflectance of at least 2.5.

24. The method for reducing the appearance of skin imperfections of claim 23, wherein the soft-focus cosmetic composition comprising:
at least one natural oil present in an amount ranging from about 0.5% to about 20%;
at least one blurring agent present in an amount ranging from 0.01% to about 20%, wherein the at least one blurring agent is selected from the group consisting of effect pigment, silica based material, polymeric material, and mixtures thereof;
at least one water-in-oil emulsifier present in an amount ranging from about 0.01% to about 20%;
wherein all percentages are based on the total weight of the soft-focus cosmetic composition;
wherein the soft-focus cosmetic composition has a diffuse reflectance of at least 2.5;
wherein the soft-focus cosmetic composition has a specular reflectance of at least 3.4;
wherein the soft-focus cosmetic composition is a water-in-oil composition.

25. The method for reducing the appearance of skin imperfections of claim 23, wherein the effect of reducing the appearance of skin imperfections is instant upon the application of the soft-focus cosmetic composition.
